(19) United States
(12) Patent Application Publication
(10) Pub. No.: US 2006/0018833 A1
(43) Pub. Date: Jan. 26, 2006

(54) Method and system for screening compounds for muscular and/or neurological activity in animals

(76) Inventors: Randall Murphy, Glenmoor, PA (US); Vincent Pieribone, New Haven, CT (US)

Correspondence Address: CANTOR COLBURN, LLP, 55 GRIFFIN ROAD SOUTH, BLOOMFIELD, CT, 06002, US

(21) Appl. No.: 11/101,151

(22) Filed: Apr. 7, 2005

Related U.S. Application Data
(60) Provisional application No. 60/560,380, filed on Apr. 7, 2004.

(51) Int. Cl. A61K 49/00

(52) U.S. Cl. 424/9.2; 800/3

(57) ABSTRACT

Screening methods and instrumentation for candidate pharmacological agents are applied to discover compounds with muscular and/or neurological activity. The method comprises the use of teleost fish, such as the medaka (Oryzias latipes), which may be stimulated with chemical agents or an electric field to produce, for example, seizure activity and/or convulsive activity. The convulsive behavior may be recorded optically and electrically. Antagonism of the convulsive behavior is produced by application of candidate pharmacological agents to the well containing the fish. The method may include stimulation and antagonism in a plurality of sample wells with a repetitive or simultaneous application of threshold electric fields. The methods and instrumentation can be applied to the study of other serious neurological diseases such as neuropathic pain. In addition the process of assaying the protection of animals to convulsant agents the assay measures pharmacological safety parameters including sedation and cognitive impairment.

### CROSS REFERENCE TO RELATED APPLICATIONS

This application claims priority from U.S. Provisional Patent Application No. 60/560,380 filed Apr. 7, 2004, which is hereby incorporated by reference in its entirety.

### FIELD OF THE INVENTION

The invention provides a system and method for screening compounds in animals for pharmacological utility. Methods of screening compounds in teleost fish by contacting fish with test agents, detecting muscular and/or neurological activity in the fish, and identifying compounds that modify muscular and/or neurological activity are provided. Certain aspects of the invention pertain to screening compounds for anti-convulsant activity in man and animals. Systems for screening compounds are also provided. Such systems include systems for automatically dispensing fish for compound evaluation and systems for detecting muscular and/or neurological activity in fish.

### BACKGROUND OF THE INVENTION

Epilepsy was one of the first neurological diseases against which drugs were effectively advanced. In 1857 the first anti-epileptic agent, bromide salt, appeared. In 1912 phenobarbital came into use and in 1938, phenytoin. The discovery and analoging of these drugs was aided early on by the development of the electroconvulsive-shock animal model of epilepsy. In the 1960s both carbamazepine and valproic acid were introduced and beginning in the 1990 there have been a range of new antiepileptic drugs introduced including felbamate, lamotrigine, gabapentin, topiramate and tiagabine. Generally, the treatment of epilepsy has thus been considered to be a major success within neurology. However, this presumption is not completely correct. Only about half of newly diagnosed patients with epilepsy obtain complete control of their seizures with the first antiepileptic drug tried, an additional 13% enter remission with a different (second) drug. The remainder of the population with epilepsy is not likely to obtain satisfactory seizure control with the use of any single drug or combination of multiple agents. This vexing problem of pharmacoresistance is familiar to all clinical epileptologists. Many practitioners assume that certain epilepsies, such as catastrophic epilepsies of childhood and some lesional epilepsies including those associated with mesial temporal sclerosis and cortical dysgenesis, are more likely to be refractory to drug treatment, perhaps because the underlying mechanisms of seizure generation in these forms of epilepsy are especially resistant to antiepileptic drugs. However, in recent years it has become evident that diagnosis and organic pathology is a priori a poor predictor of whether an individual patient will respond to treatment with a specific drug or combination of drugs. Thus, there is a major unmet medical need for new anti-convulsant compounds for the treatment of seizures, including epileptic seizures, despite the major advances which have been made in pharmacotherapy within the last ten years.

Conventional compound screening, which typically involves identifying compounds that interact with a particular “target”, usually a discrete gene or protein, is of limited utility in identifying new anti-seizure compounds. Numerous irregularities in brain biochemistry, physiology, and physical structure are know to cause seizures, including epileptic seizures, and there are still many unknown causes of seizures. A “target-independent” screening method—that is a method not dependent on the interactions of screened compounds with a particular target—is a highly desirable tool in the search for new anti-seizure drugs.

Animal Models of Epilepsy

Models of anti-convulsant drug activity in rodents, capable of identifying active anti-convulsant compounds in a target-independent fashion, exist. For example anti-convulsant activity can be predicted by administering a potential drug compound, or “test agent” to groups of mice and quantifying the compound's ability to prevent the hind limb tonic extension component of seizure when seizures are induced by maximal electroshock (MES) or administration of a known convulsant, such as pentylenetetrazol. Dilantin and phenobarbital are used as positive controls in this assay.

Such rodent assays are useful for verifying the in vivo activity of compounds identified in conventional “target-dependent” screens but cannot be used for true drug screening as the throughput then number of assays that can be performed in a day, is far too low.

Electrofishing, which has been used for over 50 years and has been heavily studied by fisheries scientists, can involve a process of applying an alternating high voltage pulse to the water surrounding a group of fish. This procedure causes a short-lived period of heightened motor activity followed by a period of ‘sleep’ during which the fish are easily netted. This sequence appears to represent a ‘tonic/clonic-type’ seizure followed by an ‘absence-type’ seizure.

Only a few studies have explored epileptogenesis, or the generation of epileptic seizures, in non-mammalian vertebrates. However it is clear that the central nervous systems of both amphibia and teleosts exhibit classical epileptiform activity. Administration of electrical stimuli, or penicillin or other chemical stimulus, has been shown to induce epileptiform electrical activity and behaviors in teleosts. Although teleosts (bony fish) and amphibia lack any laminated cortical structures these species exhibit the electrical and behavior signatures of seizures and are useful in animal models of seizure disorders, including epilepsy.

Teleosts lack many aspects of the classic mammalian cortical structures, and for this reason are an unlikely model of cortical seizures. However, there does not appear to be a significant difference between the pharmacological and physiological profile of cortical and subcortical seizures. A notable exception to this rule is glycine-mediated seizures, which likely arise from the pervasive glycinergic system present in the spinal cord. Teleost have similar glycinergic mechanisms within the spinal cord. Thus seizures in teleosts can serve as a useful model of seizures in humans and other mammals.

Some anti-seizure and targets are known to play a role in other types of muscular and/or neurological disorders. For example voltage dependent sodium channels and GABA_{A} receptors are known anti-seizure targets, but are also known to play a role in disorders such as cardiac rhythm disorders, neuropathic pain (both allodynia and hyperalgesia), attention deficit disorder, anxiety, depression, and insomnia. Thus animal models useful for identifying compounds for the treatment of seizure disorders, including epilepsy, are also useful for identifying compounds efficacious in treating other muscular and/or neurological disorders.

Existing methods of screening compounds for anti-seizure activity have two major disadvantages. The methods are either “target dependent” in vitro assays that fail to identify many efficacious compounds or very labor intensive and low throughput mammalian models. The present invention solves these problems, providing a rapid and high throughput “target independent methods and systems for identifying compounds with anti-seizure activity. The methods and system described herein are also capable of identify compounds efficacious in the treatment of a broad range of muscular and/or neurological disorders. These and other advantages of the invention are described herein.

### SUMMARY OF THE INVENTION

The inventors have found that seizures in teleost, such as the Medaka and zebrafish closely resemble seizures in humans, despite the lack of cortical structure and major differences in brain architecture and anatomy. It has been further found that anticonvulsant drugs which are active in human epilepsy attenuate seizures in teleost in a dose-dependent manner resembling that in man.

The present invention uses chemical and electrical initiated seizures in teleosts as a model of mammalian seizure disorders, including epilepsy. In this method teleost fish (e.g., Danio rerio and Oryzias latipes) are brought into contact with a potential therapeutic agent, a “test agent”, seizures are evoked via the application of either an electrical or chemical stimulus. Seizure activity is detected and usually recorded. Test agents that modify teleost's responses to electrical and/or chemical stimuli are identifying as potential therapeutic agents. Usually the system or method includes the use of a negative control—teleosts that are exposed to an electrical or chemical stimulus but are not contacted with a test agent. In this case the response of teleosts contacted with a test agent is compared to the response of the teleosts used as a negative control. The system or method may also include a positive control in which teleosts are exposed to an electrical or chemical stimulus and contacted with a known anti-seizure compound, in wherein the compound is present in sufficient concentration to block seizures.

The response may be any detectable change in response to the electrical or chemical stimulus in the presence of a “test agent” relative to the response that occurs when no test agent is present. However when screening for anti-seizure agents, compounds that inhibit, or decrease, the response to a seizure inducing stimulus will be selected as potential anti-seizure agents.

The method may be performed in a container, referred to as a “well,” capable of holding fish, water, and a test agent and capable of either holding a compound used as a chemical stimulus or able to tolerate the application of an electrical stimulus, such as an applied electric field. For example an electrical stimulus may be applied through a pair of electrodes inserted in the well and connected to a power source. In some instances the method is performed in multiwell plates.

The response may be detected by a number of means, including optical and electrical recording devices. For example an optical detector may be configured to detect light transmitted or refracted from the wells through a high transmittance portion of the well. A signal processing system is then used to interpret the significance of light transmitted or refracted from the wells. Methods and systems for detecting and recording response are described in greater detail that follows.

In one aspect the invention provides a method of identifying an agent that modifies a muscular activity, a neurological activity, or both that comprises contacting a teleost fish with a muscular stimulus, a neurological stimulus, or both, and a test agent; detecting the muscular activity, the neurological activity, or both in the teleost fish; wherein when the test agent produces a detectable change in the muscular activity, the neurological activity, or both in the teleost fish, identifying the test agent as an agent that modifies the muscular activity, the neurological activity, or both.

Also included is a method of screening that comprises providing a plurality of test wells, each test well comprising at least one teleost fish, contacting the teleost fish in at least a first fraction of the test wells with a test agent, administering a muscular stimulus, a neurological stimulus, or both to the plurality of test wells, detecting a muscular activity, a neurological activity, or both in the teleost fish; wherein when the test agent produces a detectable change in the muscular activity, the neurological activity, or both in the teleost fish in at least one test well, identifying the test agent as an agent that modifies the muscular activity, the neurological activity, or both.

Further provided herein is a screening system for identifying agents that modify a muscular activity, a neurological activity, or both comprising: a plurality of test wells each test well comprising at least one teleost fish and at least one test well comprising a test agent, a means for applying a muscular stimulus, a neurological stimulus, or both, wherein the means is sufficient to induce the muscular activity, the neurological activity, or both to the fish in the test wells; and a means for detecting the muscular activity, the neurological activity, or both in the teleost fish.

### BRIEF DESCRIPTION OF THE DRAWINGS

FIGS. 1 and 2. Flow charts of embodiments for delivery systems that deposit teleost fish into a receptacle.

FIG. 3. Schematic of an exemplary apparatus for electrical stimulation of a physiological response in a teleost fish and detection of the physiological response.

FIG. 4. Recording of induced convulsant activity in a teleost fish in real time.

FIG. 5. Detection of chemically induced seizure activity in Medaka fish.

### DETAILED DESCRIPTION OF THE INVENTION

Terminology

Prior to setting forth the invention in detail, it may be helpful to provide definitions of certain terms to be used herein. Unless otherwise stated standard nomenclature is used. Unless defined otherwise, all technical and scientific terms used herein have the same meaning as is commonly understood by one of skill in the art to which this invention belongs.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “muscular and/or neurological stimulus” refers to an external stimulus that is capable of inducing a muscular and/or neurological change in a teleost fish. Suitable muscular and/or neurological stimuli include electrical stimuli and chemical stimuli.

Certain test agents have an effect on muscular and/or neurological activity. The term “muscular and/or neurological activity” refers to the muscles and/or nerves the test agents stimulate including, for example, skeletal muscles and cardiac muscle. In one embodiment, a test agent affects neuromuscular activity, i.e., both muscular and neurological activity. Muscular and/or neurological activity includes seizure and anti-seizure activity (including convulsant and anti-convulsant activity), cardiac rhythm activity, pain aversive activity (e.g., compounds that affect the perception of pain also called nociception), and the like. Agents that modify a biological activity, such as muscular and/or neurological activity may be antagonists, agonists, or inverse agonists.

An agent is considered an “antagonist” if it detectably decreases a response to a neurological and/or muscular stimulus. For example an agent that produce a response to a neurological and/or muscular stimulus that is significantly less than the response to the same stimulus in the absence of the agent is an antagonist. Similarly an “agonist” as used herein is an agent that detectably increase a response to a neurological and/or muscular stimulus. For example an agent that produce a response to a neurological and/or muscular stimulus that is significantly greater than the response to the same stimulus in the absence of the agent is an agonist.

An “inverse agonist” is an agent that reduces detectably activity reduces a response to a neurological and/or muscular stimulus below the activity level observed in the absence of the stimulus.

A “detectable change” is any change that is greater than the minimal detectable change in the detection system used. In certain circumstances a detectable change is a change that is detected as a signal with a signal to noise ratio of at least 3 to 1. When the detectable change is a “difference” the difference may be any quantifiable or discernable difference between two data points. However when determining whether a test agent modifies an activity it is preferred that the difference is a statistically significant difference with results varying from control at the p<0.1 level or more preferably at the p<0.05 level of significance as measured using a standard parametric assay of statistical significance such as a student's T test.

The term “electrical stimulation” refers to an electric current or shock applied to a responsive tissue. Electrical stimulation includes a voltage change in a well containing a teleost fish. Such a voltage change across a well may produce a voltage change in the in cells of the fish central nervous system resulting in, for example, a convulsion. Electrical stimulation can involve a fixed voltage, current or power, a range of frequencies (1-200 Hz), unipolar or bipolar with varied duty cycle, square waves, triangle waves or sine waves.

The term “electrode” means a conductor used to establish electrical contact with a nonmetallic part of a circuit. Electrodes include conductors made from tungsten, silver, silver/silver chloride, platinum, palladium, stainless steel, iridium, copper, silicon based conductive materials, and combinations comprising one or more of the forgoing electrode materials. The electrodes may be discrete or in a printed circuit package, insulated or bare.

The term “A-to-D” means analog to digital.

The term “multiwell plate” refers to a two dimensional array of wells located on a substantially flat surface. Multiwell plates may comprise any number of discrete wells, and may comprise wells of any width or depth. Wells are either uncovered or openable so that materials, such as liquid reagents may be added. Common examples of multiwell plates include 6, 12, 24, 48, 96, 384, and 3456 well plates. Such multiwell plates may be constructed of plastic, glass, a mixture of both, or any essentially electrically nonconductive material.

A “resting potential” for a cell is equilibrium transmembrane potential of a cell not subjected to external influences.

A “seizure” is a sudden attack or convulsion usually due to an uncontrolled burst of electrical activity in the brain that can result in a wide variety of clinical manifestations such as: muscle twitches, staring, tongue biting, urination, loss of consciousness and total body shaking. A “convulsion” is a violent involuntary contraction or series of contractions of the voluntary muscles.

A “teleost” is a fish belonging to the taxa group Telostei or Teleostomi. The teleost include numerous fishes having bony skeletons and rayed fins, for example, zebrafish (Danio rerio, Danio rerio), medaka (Oryzias sp.), fathead minnows (Pimephales promelas), and goldfish (Carassius auratus). Suitable teleosts include, for example, Oryzias latipes, Astronotus ocellatus, Danio rerio, Anguilla anguilla, Chelon labrosus, Salmo truttafario, Oncorhynchus mykiss, Oreochromis mossambicus, Eigenmannia virescens, Cyprinus carpio, Stephanolepis cirrhifer, Carassius auratus, Gasterosteus aculeatus, Clarias batrachus, Pimephales promelas, Apteronotus leptorhynchus, and combinations comprising one or more of the foregoing teleosts.

The term “test agent” refers to a chemical or natural product, usually a small molecule organic compound of less than 700 MW, to be tested by a screening method as a putative modulator. A test agent can be a chemical, such as an inorganic chemical, a charged ion, an organic chemical, a protein, a nucleic acid (single or polymerized), a peptide, a carbohydrate, a lipid, or a combination comprising one or more of the foregoing. Usually, various predetermined concentrations of test agents are used for screening, such as, for example, 10 nM, 0.1 μM, 1 μM and 10 μM.

The term “threshold electrical stimulation” means initiating a voltage change in well containing a teleost fish, wherein such a voltage change produces a voltage in cells of the fish central nervous system resulting in a convulsion, wherein the voltage is the minimal required to produce a convulsion or provide a painful stimulus.

The term “transistor-transistor logic” or “TTL” refers to an electronic logic system in which a voltage around +5V is TRUE and a voltage around 0V is FALSE.

A “uniform electric field” is an electric field varies by no more than 15% from the intended mean intensity within the observation area at any one time.

Screening Test Agents

Methods of Screening Test Agents for Neurological and/or Muscular Activity Include contacting a second teleost fish with a muscular stimulus, a neurological stimulus, or both; detecting a second muscular activity, neurological activity, or both in the second teleost fish;
wherein when the test agent produces a detectable change that is a difference between in the muscular activity, the neurological activity, or both in the teleost fish contacted with the test agent and the second teleost fish, identifying the test agent as an agent that modifies the muscular activity, the neurological activity, or both.

The muscular stimulus, the neurological stimulus, or both, comprises an electrical stimulus, a chemical stimulus, or both.

Detecting the muscular activity, the neurological activity, or both may comprises detecting a behavioral response in the teleost fish. The behavioral response is rapid and erratic tail movements, or post-ictal inactivity.

The detection step in the screening methods disclosed herein may include recording the muscular activity, the neurological activity, or both in real time.

Also provided herein is a s method of screening test agents for neurological and/or muscular activity by providing a plurality of test wells, each test well comprising at least one teleost fish, contacting the teleost fish in at least a first fraction of the test wells with a test agent, administering a muscular stimulus, a neurological stimulus, or both to the plurality of test wells, detecting a muscular activity, a neurological activity, or both in the teleost fish; wherein when the test agent produces a detectable change in the muscular activity, the neurological activity, or both in the teleost fish in at least one test well, identifying the test agent as an agent that modifies the muscular activity, the neurological activity, or both. This methods may additionally include a subset of the plurality of test wells, a second fraction of test well in which the fish in the second fraction of test wells are not contacted with a test agent, wherein when the test agent produces a detectable change that is a difference between in the muscular activity, the neurological activity, or both in the teleost fish in the first fraction of test wells and the muscular activity, the neurological activity, or both in the teleost fish in the second fraction of test wells, identifying the test agent as an agent that modifies the muscular activity, the neurological activity, or both.

The means for detecting the muscular activity and/or the neurological activity may comprise a plurality of photodetectors in communication with a plurality of photoemitters.

In the screening methods described herein the muscular stimulus, the neurological stimulus, or both is an electric field, a chemical stimulus, or both.

The detection means for detecting the muscular activity, the neurological activity or both may comprise: a plurality of photodetectors capable of detecting convulsant activity in the fish, wherein the photodetectors are situated substantially adjacent to a side of each well; and a plurality of photoemitters situated substantially adjacent to a side of each well substantially opposing the array of photodetectors array; and a means for recording the convulsant activity in real time.

In the screening methods described herein the muscular stimulus, the neurological stimulus, or both is a convulsant stimulus, the detected muscular activity, neurological activity, or both is a convulsant activity, and the test agent inhibits the convulsant activity. In other embodiments the neurological stimulus, or both is a neuropathic pain stimulus, the detected muscular activity, neurological activity, or both is a pain aversive activity, and the test agent inhibits the pain aversive activity. In still other embodiments the muscular stimulus, the neurological stimulus, or both is a cardiac rhythm stimulus, the detected muscular activity, neurological activity, or both is a cardiac rhythm, and the test agent modifies the cardiac rhythm.

Screening Systems

In addition to the screening system described above, other embodiments described herein include a screening system that comprises a means for comparing the muscular activity, the neurological activity, or both in the teleost fish in the presence of a test agent to a control muscular activity, neurological activity, or both in the teleost fish in the absence of the test agent. The means for applying the muscular stimulus, neurological stimulus, or both is a pair of inert conductive electrodes capable of applying an electric field to the test wells.

Certain screening systems described herein include a plurality of tests wells in the form of a multiwell plate, a means for applying a convulsive stimulus sufficient to induce convulsive activity in the fish in the test wells, a means for applying a neuropathic pain stimulus sufficient to induce pain adversive activity in the fish in the test wells, or a cardiac rhythm stimulus sufficient to modify cardiac rhythm in the fish in the test wells wherein the stimulus is either an electric field or a convulsive chemical.

The means for detecting muscular and/or neurological activity in the fish comprises: may be a plurality of photodetectors capable of detecting a muscular activity, a neurological activity, or both in the fish, wherein the photodetectors are situated substantially adjacent to a side of each well; and a plurality of photoemitters situated substantially adjacent to a side of each well substantially opposing the array of photodetectors; and a means for recording the muscular activity, the neurological activity, or both, in real time, wherein the muscular activity or neurological activity is a convulsive activity, a cardiac rhythm, or a pain adversive activity.

The screening system may additionally comprise a means for comparing convulsive activity in the fish in the presence of a test agent to convulsive activity in the fish in the absence of the test agent.

The screening system may comprise a means for comparing the convulsant activity in the fish in the presence of a test agent to convulsant activity in the fish in the absence of the test agent.

Selection and Maintenance of Animals

The method and screening systems described herein can measure the effect of a “test agent” in modifying the effect of an electrical or chemical stimulus in a variety of small fish and non-fish species. The effect of a test agent on the response of fish to an external neurological and/or muscular stimulus may be indicative of the response in humans or other mammals. Two species of teleost fish; zebrafish (Danio rerio) and the Japanese rice fish Medaka (Oryzias latipes); are particularly useful in the disclosed method and system. Both species have distinct advantages. An advantage of the Medaka (Oryzias latipes) is that a complete genomic sequence is available. Also, an almost completely transparent strain of Medaka (Oryzias latipes) exists. An advantage of zebrafish is that female zebrafish can lay between 200-400 eggs at a time. Medaka, in contrast, produce around 7-20 at a time. Zebrafish eggs hatch on a much quicker and more consistent schedule, 40-48 hours after being layed. Medaka incubate 6-8 days, but emerge in a much more mature state. Zebrafish have more difficult feeding requirements as fry while Medaka can be supported immediately upon birth on brine shrimp.

Transgenic teleosts may also be employed. The term “transgenic” is used to describe an organism that includes exogenous genetic material (a transgene) within all of its cells. The term includes an organism whose genome has been altered by in vitro manipulation of the early embryo or fertilized egg or by a transgenic technology to induce a specific gene knockout. The term “transgene” refers a piece of DNA, which is inserted by artifice into a cell, and becomes part of the genome of the organism (i.e., either stably integrated or as a stable extrachromosomal element), which develops from that cell. Such a transgene may include a gene, which is partly or entirely heterologous (i.e., foreign) to the transgenic organism, or may represent a gene homologous to an endogenous gene of the organism. Included within this definition is a transgene created by the providing of an RNA sequence that is transcribed into DNA and then incorporated into the genome. Transgenes include DNA sequences that encode a fluorescent or bioluminescent protein that may be expressed in a transgenic non-human animal.

In some embodiments, the transgenic teleost may comprise a gene knockout. The term “gene knockout”, as used herein, refers to the targeted disruption of a gene in vivo, which results in loss of function of that gene. A gene knockout may be achieved by any transgenic technology familiar to those in the art. In one embodiment, transgenic animals having gene knockouts are those in which a target gene has been rendered nonfunctional by an insertion targeted to the gene to be rendered non-functional by homologous recombination. Transgenic animals comprising gene knockouts may be particularly useful in determining the mechanism of action of test agents.

Multiwell Plates

For the purposes of screening, it may be convenient to perform multiple assays substantially simultaneously. One or fish may be deposited in at least one well of a multiwell plate. Tissue culture assemblies, commonly referred to as tissue culture plates or multiwell plates, are used for in vitro cultivation of cells particularly for experimental purposes. In order to increase throughput, multiwell tissue culture plates have been designed, which include 6, 12, 24, 48, 96, and 384, and more wells. Such multiwell tissue culture plates allow the investigators to use semi- or fully-automated devices to conduct tests of many individual cell cultures at a speed unachievable by regular tissue culture assemblies.

Multiwell tissue culture assemblies are exemplified in U.S. Pat. Nos. 4,349,632; 4,038,149; 4,012,288; 4,010,078; 3,597,326 and 3,107,204, which are hereby incorporated by reference for their teachings regarding multiwell plates. Other culture vessels are exemplified in U.S. Pat. No. 4,358,908 and U.S. Pat. No. 4,657,867, which are hereby incorporated by reference for their teachings regarding multiwell plates.

Suitable multiwell plates are designed primarily to provide for efficient electrical stimulation of cells while at the same time enabling the optical analysis of neurological or muscular changes in the test organism. To accomplish these goals, conductive surface electrodes may be orientated in, or on, the walls, bottoms or lids of the multiwell plate. In general such multiwell plates can have a footprint of a suitable shape or size, such as square, rectangular, circular, oblong, triangular, kidney, or other geometric or non-geometric shape. The footprint can have a shape that is substantially similar to the footprint of existing multiwell plates, such as the standard 48-well microtiter plate, whose footprint is approximately 85.5 mm in width by 127.75 mm in length, or other sizes that represent a current or future industry. Multiwell plates of the present invention having this footprint can be compatible with robotics and instrumentation, such as multiwell plate translocators and readers as they are known in the art.

Typically, wells will be arranged in two-dimensional linear arrays on the multiwell plate. However, the wells can be provided in any type of array, such as geometric or non-geometric arrays. The multiwell plate can comprise a suitable number of wells. Commonly used numbers of wells include 6, 12, 96, 384, 1536, 3456, and 9600.

Well volumes can vary depending on well depth and cross sectional area. In one embodiment, the well volume is about 0.1 microliters to about 10,000 microliters. Wells can be made in a suitable cross sectional shape (in plan view) including, square, round, hexagonal, other geometric or non-geometric shapes, and combinations (intra-well and inter-well) thereof. Suitable multiwell plates have square or round wells, with flat bottoms.

The walls of the multiwell plate can be chamfered (e.g. having a draft angle). Specifically, the angle is about 1 to about 10 degrees, more preferably about 2 to about 8 degrees, and most preferably about 3 to about 5 degrees.

The wells can be placed in a configuration so that the well-center to well-center distance is about 0.5 millimeters to about 100 millimeters. The wells can be placed in any configuration, such as a linear-linear array, or geometric patterns, such as hexagonal patterns. The well-to-well distance can be about 9 mm for a 96 well plate. Smaller well-center to well-center distances are preferred for smaller volumes.

The wells can have a depth of about 0.5 to about 100 millimeters. Preferably, the well depth is about 1 millimeter to about 100 millimeters, more preferably about 2 millimeters to about 50 millimeters, and most preferably about 3 millimeters to about 20 millimeters.

The wells can have a diameter (when the wells are circular) or maximal diagonal distance (when the wells are not circular) of about 0.2 to about 100 millimeters. Preferably, the well diameter is about 0.5 to about 100 millimeters, more preferably about 1 to about 50 millimeters, and most preferably about 2 to about 20 millimeters.

In one embodiment, each well also comprises a bottom having a high transmittance portion. Preferably, the bottom is a plate or film as these terms are known in the art. The thickness of the bottom can vary depending on the overall properties required of the plate bottom that may be dictated by a particular application. Well bottom layers may have a thickness of about 10 micrometers to about 1000 micrometers. In certain embodiments the well bottom has a thickness of about 10 μm to about 450 μm, about 15 μm to about 300 μm, or about 20 μm to about 100 μm.

The bottom of a well can have a high transmittance portion, meaning that either all or a portion of the bottom of a well can transmit light. The bottom can have an optically opaque portion and a high transmittance portion of any shape, such as circular, square, rectangular, kidney shaped, polygonal, or other geometric or non-geometric shape any combinations of the foregoing.

In one embodiment, the bottom of the multiwell plate can be substantially flat, e.g. having a surface texture of about 0.001 mm to about 2 mm, preferably about 0.01 mm to about 0.1 mm, according to ANSI standards B46.1-2985. If the bottom is not substantially flat, then the optical quality of the bottom and wells can decrease because of altered optical and/or physical properties.

The materials for manufacturing the multiwell plate may be polymeric, since these materials lend themselves to mass manufacturing techniques. The top and the bottom can be made of the same or different materials. The bottom of the multiwell plate may comprise glass or quartz. Suitable polymers may have low fluorescence and/or high transmittance. Polymeric materials can particularly facilitate plate manufacture by molding methods known in the art such as insert or injection molding.

The multiwell plate can be made of one or more pieces. For example, the plate and bottom can be made as one discrete piece. Alternatively, the plate can be one discrete piece, and the bottom can be a second discrete piece, which are combined to form a multiwell plate. In this instance, the plate and bottom can be attached to each other by sealing means, such as adhesives, sonic welding, heat welding, melting, insert injection molding or other means known in the art or later developed. The plate and bottom can be made of the same or different material. For example, the plate can be made of a polymer, and the bottom made of polystyrene, cycloolefin, glass, or quartz.

Automatic Distribution of Fish to Multiwell Plate Wells

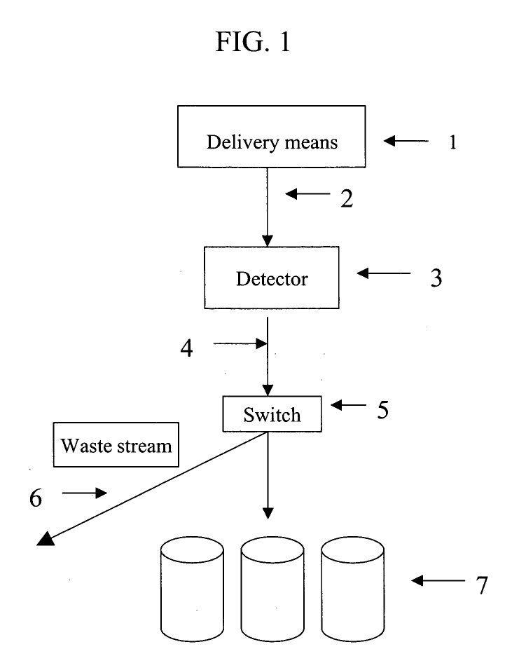

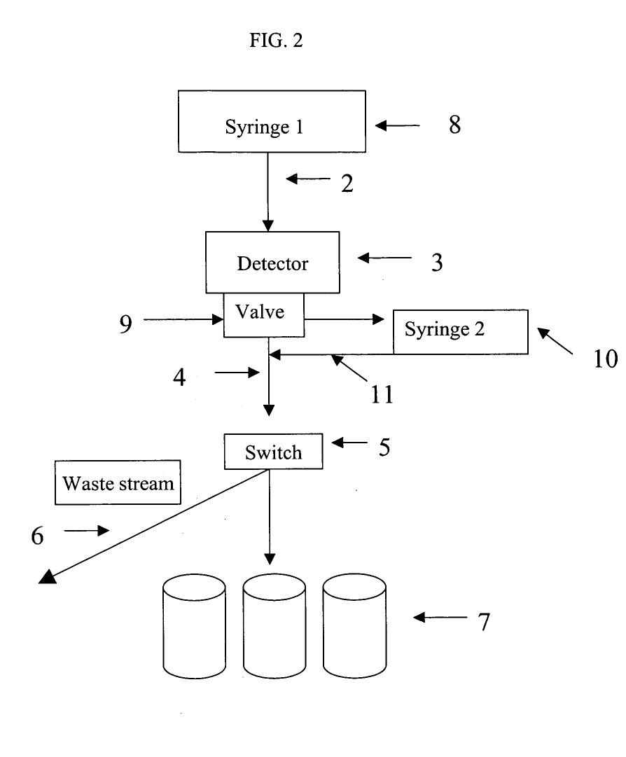

In one embodiment, a large number of animals may be assayed rapidly. Disclosed is a method for rapidly and gently placing a single (or several) fish into each well in an automated fashion to create the multiwell plates suitable for use, for example, in a screening robot. A fluidics device capable of delivering fish into wells automatically may be employed (FIG. 1). Such a device comprises a means for delivering (1) a carrier stream (2) comprising a carrier fluid and a plurality of fish in fluid communication with a detector (3). The means for delivery (1) includes for example, gravity-feed, a pump, a syringe, or other suitable fluid delivery device. The efflux stream (4) from the detector (3) is in fluid communication with a switch (5) in fluid communication with a waste stream (6) and a receptacle for the fish (7). The switch (5) can switch the efflux stream (4) from between a waste stream (6) and a receptacle (7) for the fish. Upon detection of a fish in the efflux stream (5), the efflux stream (5) is temporarily diverted from a waste stream (6) into a well or other receptacle (7) for the fish allowing for the deposition of one or more fish into the receptacle.

In one embodiment, the means for delivery (1) comprises a 50 ml syringe pump (syringe 1) (8) (Harvard) and the carrier stream (2) comprises a high concentration of fish fry (e.g., at least about 5 per ml) in a carrier fluid (e.g., water). The carrier stream (2) comprising fish is ejected from the syringe (8) at a moderate rate (typically 1 ml per 15 seconds). In this embodiment, as fish enter the tubing connected to the syringe, their movement is retarded by the flow of liquid. The detector (3) comprises a photointerrupter circuit and a pinch valve. As a fish passes the photointerrupter circuit, it triggers the detector (3) to shut a pinch valve (9), which the fish just passed, opening a valve from syringe 2 (10) that contains only carrier fluid (11) (e.g., water). In this manner, the pinch valve (9) can be used to isolate the desired number of fish. In some embodiments, a single fish will be isolated. As the pinch valve (9) is switching, a second set of down stream switches (5) are activated. Prior the isolation of a fish by the pinch valve, the efflux stream (4) is directed to waste (6). Upon isolation of a fish by the pinch valve (9) and opening of the valve for syringe 2 (10), the efflux stream (4) is redirected to a receptacle (7). Adequate water may be passed into the well from syringe 2 (10) to ensure that the fish has been deposited in the receptacle (7). The efflux stream is directed to the receptacle for a time sufficient to allow for the deposition of a fish. The amount of water deposited into the well may be controlled so each well has about the same depth of water. A consistent water level can ensure that the electrical stimulation is consistent. After deposition of the fish to the receptacle, switch (5) is activated such that the efflux stream (4) is again directed to waste (6) and the cycle can be repeated. If the receptacle is a well of a multiwell plate, either the multiwell plate or the efflux stream may be translated such that a second well of the multiwell plate can receive a fish.

Automatic Distribution of Compounds into Multiwell Plates

Automatic distribution of compounds into a multiwell plate can, for example, be performed in a device that integrates a liquid handler operably linked to a multiwell positioning stage. In one embodiment, the liquid handler comprises a dispensing nozzle. In one embodiment, the liquid handler can comprise a plurality of pipetting tips that can individually dispense a predetermined volume. Typically, pipetting tips are arranged in two-dimension array to handle multiwell plates of different well densities (e.g., 96, 384, 864 and 3,456).

The dispensed volume may be aspirated from a predetermined selection of addressable wells and dispensed into a predetermined selection of addressable wells. A microliter or nanoliter pipetting tip (or smaller volume dispenser) comprises a fluid channel to aspirate liquid from a predetermined selection of addressable wells (e.g., chemical wells containing drug candidates).

In one embodiment, pipetting tips (e.g., nanoliter pipetting tips) comprise solenoid valves fluidly connected to a reservoir for liquid from an addressable well. The fluid reservoir can be a region of a dispenser that can hold fluid aspirated by the pipetting tip. Typically, a tip reservoir will hold at least about 100 times the minimal dispensation volume to about 10,000 times the dispensation volume and more specifically about 250,000 times the dispensation volume. The solenoid valves control a positive hydraulic pressure in the reservoir and allow the release of liquid when actuated. A positive pressure for dispensation can be generated by a hydraulic or pneumatic means, e.g., a piston driven by a motor or gas bottle. A negative pressure for aspiration can be created by a vacuum means (e.g., withdrawal of a piston by a motor). For greater dispensing control, two solenoid valves or more can be used where the valves are in series and fluid communication.

In another embodiment, pipetting tips (e.g., nanoliter pipetting tips) comprise an electrically sensitive volume displacement unit in fluid communication to a fluid reservoir. Typically, the fluid reservoir holds liquid aspirated from an addressable well. Electrically sensitive volume displacement units are comprised of materials that respond to an electrical current by changing volume. Typically, such materials can be piezo materials suitably configured to respond to an electric current. The electrically sensitive volume displacement unit is in vibrational communication with a dispensing nozzle so that vibration ejects a predetermined volume from the nozzle. Piezo materials may be used in dispensers for volumes less than about 10 to 1 nanoliter, and are capable of dispensing minimal volumes of 500 to 1 picoliter. Piezo pipetting tips can be obtained from Packard Instrument Company, Connecticut, USA (e.g., an accessory for the MultiProbe 104). Such small dispensation volumes permit greater dilution, conserve and reduce liquid handling times.

In some embodiments, the liquid handler can accommodate bulk dispensation (e.g., for washing). By connecting a bulk dispensation means to the liquid handler, a large volume of a particular solution to be dispensed many times. Such bulk dispensation means are known in the art and can be developed in the future.

Interrogation, aspiration or dispensation into multiwell plates can be accomplished by automated positioning (e.g. orthogonal) of a multiwell plate. The multiwell plates may be securely disposed on an orthogonal positioner that moves the wells of a multiwell plate with a first density in an X,Y position with respect to the X,Y position of the liquid handler. The liquid handler may have an array of aspiration and/or dispensation heads, or both. Many aspiration/dispensation heads can operate simultaneously. The orthogonal positioner will align each addressable well with the appropriate dispensing head. A predetermined location (e.g., center) of a pre-selected addressable well may be aligned with the center of a dispensing head's fluid trajectory. Other alignments can be used, such as those described in the examples. With a head substantially smaller than a well diameter, orthogonal positioning permits aspiration or dispensation into plates of different densities and well diameters.

An orthogonal positioner can typically match an array of dispensing heads with an array of addressable wells in X,Y using a mechanical means to move the addressable wells into position or the liquid handler (e.g., dispensing heads) into position. Arrays of addressable wells on a plate may be moved rather than the liquid handler. This design often improves reliability, since multiwell plates are usually not as heavy or cumbersome as liquid handlers, which results in less mechanical stress on the orthogonal positioner and greater movement precision. It also promotes faster liquid processing times because the relatively lighter and smaller multiwell plates can be moved more quickly and precisely than a large component. The mechanical means can be a first computer-controlled servo motor that drives a base disposed on a X track and a second computer-controlled servo motor that drives a Y track disposed on the X track. The base can securely dispose a multiwell plate and either a feedback mechanism or an accurate Cartesian mapping system, or both that can be used to properly align addressable wells with heads. Other such devices, as described herein, known in the art or developed in the future to accomplish such tasks can be used. It may be desirable that such devices comprise detectors to identify the addressable wells or multiwell plates being orthogonally positioned. Such positioners for predetermined X, Y coordinates can be made using lead screws having an accurate and fine pitch with stepper motors (e.g., Compumotor Stages from Parker, Rohnert Park, Calif., USA). Positioners (e.g. X, Y or Z) can be used to move the detector assembly, the sample, liquid handler or a combination there of.

Alternatively, the liquid handler can be disposed on a Z-positioner, having an X,Y positioner for the liquid handler in order to enable precise X,Y and Z positioning of the liquid handler (e.g., Linear Drives of United Kingdom).

A reference point or points (e.g., fiducials) can be included in the set up to ensure that a desired addressable well is properly matched with a desired addressable head. For instance, the multiwell plate, the orthogonal positioner or the liquid handler can include a reference point(s) to guide the X,Y alignment of a plate, and its addressable wells, with respect to the liquid handler. For example, the liquid handler has a detector that corresponds in X,Y to each corner of a plate. The plate has orifices (or marks) that correspond in X,Y to the liquid handler's position detectors. The plate's orifices allow light to pass or reflect from a computer-controlled identification light source located on the orthogonal positioner in the corresponding X,Y position. Optical locators known in the art can also be used in some embodiment. Detection of light by the liquid handler emitted by the orthogonal positioner verifies the alignment of the plates. Once plate alignment is verified, aspiration or dispensation can be triggered to begin. Stepper motors can be controlled for some applications.

The liquid handler may optionally be disposed on a Z-dimensional positioner to permit adjustments in liquid transfer height. This feature may allow for a large range of plate heights and aspirate and dispense tips, to be used in the sample distribution module. It also permits the dispense distance between an addressable well surface, or liquid surface in an addressable well, and a liquid handler to be adjusted to minimize the affects of static electricity, gravity, air currents and to improve the X,Y precision of dispensation in applications where dispensation of a liquid to a particular location in an addressable well is desired. Alternatively, multiwell plates can be positioned on a Z-dimensional positioner to permit adjustments in liquid transfer height. Static neutralizing devices can also be used to minimize static electricity. Generally, the liquid transfer height will be less than about 2 cm. Specifically, small volumes may be dispensed at a liquid transfer height of less than about 10 mm, and more preferably less than about 2 mm. Occasionally, it may be desirable to contact the tips with a solution in a controllable fashion, as described herein or known in the art.

A commercially available apparatus to enable automatic distribution is available from Beckman Instruments, Caliper Technologies, Newport Industries, Thorlabs Inc. and Robbins industries Inc.

Electrodes and Electrode Arrays for Neurological and/or Muscular Stimulus

In one embodiment the means for administering a neurological and/or muscular stimulus to all or a fraction of the test wells includes electrodes and electrode arrays, for creating electrical fields across the area of observation. Stimulation may be achieved by a single, repetitive, or simultaneous application of threshold electric fields. Electrical fields can be achieved via a pair of electrically conductive electrodes. One design feature is that the electrode pairs create well-defined electrical fields. Suitable electrode designs include electrode configurations that maximize the electric field homogeneity within the well. Field uniformity over a fixed area can be described in two ways: (1) the standard deviation of the field magnitude divided by the average field magnitude in the area, and (2) the difference between the highest and lowest field magnitudes, normalized to the average field magnitude in the area. The simplest way to generate a uniform electric field in a conductive medium is to use two substantially identical, flat electrodes with surfaces that are aligned substantially parallel to each other.

The type of electrical stimulus applied has several variables that are adjusted differently for slightly different assays: duration of electrical pulse, current fixed, voltage fixed, power fixed, frequency of the pulse, polarity of the pulse, and magnitude of electrical pulse. Electrodes are designed to ensure the most consistent power delivery and electrical field into the well. The electrical properties (resistance and capacitance) of the well are determined with the stimulation electrode, prior to the test, to estimate the impedance and inductive properties of the well.

Typical round multiwell plate wells however may constrain the width of electrodes that can be inserted into the wells, and also introduce other effects which reduce field uniformity. The roundness of the wells may provide a challenge to create a uniform field pointing in one direction with two electrodes the width of the water between the electrodes is constantly changing. Additionally the high surface tension of water may generate variations in the height of the saline across the well when dipper electrodes are inserted. The curved surface, or meniscus, can perturb the electric field throughout the volume of the well. A depth of 100 μL of saline in a 96-well plate is normally about 3.0 mm deep at the center and about 2.9 mm deep at the edges of the well. Improved electrode designs and systems for electrical stimulation address these issues to create substantially uniform electrical fields over the area of observation. In one embodiment, the electrode pair comprises two substantially parallel wire electrodes comprising an electrical insulator that is attached to the pair of electrodes to restrict current flow to a defined region thereby creating a highly uniform electrical field.

A protocol to produce stimulation of the well comprises rapidly switching amongst a plurality of (six or more) electrodes placed around the perimeter of the well. This is similar to the scrambling electrode arrays used in rodent operant boxes. By rapidly (approximately greater than 100 Hz) and semi-randomly switching the active electrodes, the voltage field is randomized and therefore more similar from well to well and animal to animal. Finally if the animal contacts the metal of the electrodes the shock delivered is greater than that experienced when tank water is in between.

Electrode design can reduce the likelihood that the animal will come in direct contact the conductive part of the electrode, exposing it to a greater than average electric current, and insures that the animal is exposed to current flow through the water. A variety of electrode shielding arrangements may be employed allowing electrical conductivity and yet shielding the animal from contact. A suitable method of allowing shielding is to cover the electrodes with fine (>600 mesh) Teflon mesh cloth. This prevents physical contact of the fish with the wire electrodes.

Suitable electrically conductive materials can be used as an electrode. Preferred electrode materials have many of the following properties, (1) they do not corrode in saline, (2) they are inert and do not produce or release toxic ions, (3) they are flexible and strong, (4) they are relatively inexpensive to fabricate, (5) they are non porous, and (6) they are easily cleaned. Suitable materials include noble metals (including gold, platinum, and palladium), refractory metals (including titanium, tungsten, molybdenum, and iridium), corrosion-resistant alloys (including stainless steel), carbon or other organic conductors (including graphite various conductive polymeric materials), and combinations comprising one or more of the foregoing materials. For most embodiments, stainless steel (340) provides an appropriate electrode material. This material is inexpensive, easy to machine, and very inert in saline. Stainless steel oxidizes slowly to produce iron oxide when passing current in saline, but this does not appear to affect the performance of the system. Iron oxide has very low solubility in water and toxic levels of iron do not appear to be released. Additionally any iron oxide deposits can easily be removed by soaking the electrodes in 10% nitric acid in water for two hours, then rinsing thoroughly with distilled water. Electrolysis products can be contained or eliminated by coating the surfaces of the electrodes with protective coatings, such as gelatin, polyacrylamide, or agarose. Dipper electrodes typically consist of one or more pairs of electrodes that are arranged in an array that can be retractably moved into, and out of, one or more wells of a multiwell plate. Dipper electrodes may be orientated into arrays that match the plate format and density, but can be in arrays of any configuration or orientation. For example for a standard 48 well plate, a number of electrode configurations are possible including electrode array arrangements to selectively excite one or more columns, or rows, simultaneously. The entire array of electrodes may be held in correct registration by a rigid non conductive member that keeps each electrode pair correctly spaced to accurately match a standard 48 well plate layout. The non-conductive member enables the electrodes to move up or down while precisely maintaining their registration with the multiwell plate.

To provide for correct registration of the electrode array with a multiwell plate, the electrode assembly can optionally comprise an outer border or flange that can accommodate a standard 48-well plate, and enables accurate plate registration. In some embodiments the border further includes a registration notch or indentation to provide unambiguous plate registration. In one embodiment the electrode array further comprises means for retractably inserting the electrode array into the wells of the multiwell plate.

In one embodiment, the electrode array further comprises an upper, movable support member to which the electrodes are attached. The movable support member is able to move up or down relative to the non-conductive member by sliding on a plurality of alignment pins. A pneumatic cylinder enables the movable support layer to automatically return to the upper position.

Electrical Means for Inducing a Stimulus

Suitable stimulation parameters include the type of waveform that may be applied to the well (i.e. sinusoidal, triangular, squarewave, bi- or monopolar), whether the stimuli should be current or voltage-limited and the shape (pulse width and duty cycle). Classically, both current and voltage-limited stimuli trains have been used with either square or sinusoidal waveforms. In one embodiment, constant current is applied to each well. Towards this end, arrays of high voltage constant current ICs (LR8) from Supertex, Inc may be employed. These circuits allow regulated constant current pulses to be given to wells. Bipolar current pulses may be directed to specific wells via digitally addressable and isolated high-voltage analog switches (HV209; Supertex, Inc.).

Methodologically, a physiological baseline may be determined for each well. Threshold electrical stimulation may be applied to evoke a seizure from the fish(s) in each well and physiological monitoring and quantification of the seizure-related motion events is performed.

Chemical Means for Neurological and/or Muscular Stimulus

Chemical means may be employed to induce neurological and/or muscular stimulation. In one embodiment, a chemical means is employed to induce seizure. A variety of chemical compounds are known to evoke seizures. These include, but are not limited to pentylenetetrazol, kainic acid, picrotoxin, biccucline, fluroethyl, and strychnine. In general agents that block the inhibitory neurotransmission mediated by GABA or glycine or enhance excitatory glutamate neurotransmission are proconvulsants.

Types of Neurological and/or Muscular Stimulus

A convulsion is an abnormal involuntary contraction or series of contractions of the muscles, also known as a seizure. In one aspect, test agents may be screened for anti-convulsive activity. In this embodiment, the muscular stimulus, the neurological stimulus, or both is a convulsant stimulus, the detected muscular activity, neurological activity, or both is a convulsant activity, and the test agent inhibits the convulsant activity. Suitable convulsant stimuli include, for example, electrical stimuli and chemical compounds known to induce seizures as described above.

Neuropathic pain is pain resulting from a disturbance in function or pathological change in a sensory nerve. In another embodiment, the muscular stimulus, the neurological stimulus, or both is a neuropathic pain stimulus, the detected muscular activity, neurological activity, or both is a pain aversive activity, and the test agent inhibits the pain aversive activity.

Pain may be induced in test subjects either by physical or chemical means. Mechanical means include but are not limited to lasers, acoustic, microwave, X-ray, impaling or crushing objects, hot objects, or induced magnetic or electrical fields. Animals may be also be exposed to a range of innocuous photocaged noxious compounds (i.e. photocaged formaldehyde) and the laser used to uncage, activate or photoreact the compound. These compounds include compounds capable of producing noxious substances such as free-radicals, amino cross-linking agents and capsicum agents known in mammalian models to induce a painful stimuli. These agents may produce tissue damage, directly activate pain nerve fibers, produce pH, redox or temperature changes when activated by the laser. In practice mostly UV lasers are used for such experiments. In another embodiment, a chemical agent is added to the well, which induces pain in the animal without additional photo activation. Such chemicals also may produce pH, redox or temperature changes in the animal. A chemical may also directly active a neuronal pain sensing system (i.e. substance P) or produce tissue damage (i.e. formaldehyde).

In the case of laser-mediated painful stimulation, the latency-to-escape reaction of the fish is quantified. At which point in the increasing laser intensity the animal initiates an escape reaction is determined and indicates the level of anti-nociception the compound affords. For chemical induced pain, the animal is exposed to progressively greater levels of the noxious chemical and the animal's behavior quantified following each addition.

Cardiac rhythm refers to the heart rate or rhythm. Arrhythmias, or disorders of the heart rhythm, are caused by a disruption of the normal electrical conduction system of the heart. Arrhythmias include, for example, ventricular fibrillation and ventricular tachycardia. Antiarrhythmic medications include medications to speed up or slow down the heart rate, and others. In yet another embodiment, the muscular stimulus, the neurological stimulus, or both is a cardiac rhythm stimulus, the detected muscular activity, neurological activity, or both is a cardiac rhythm, and the test agent modifies the cardiac rhythm. Suitable cardiac rhythm stimuli include, for example, electrical stimuli that are sufficient to induce a change in the cardiac rhythm of a teleost fish.

Physiological Monitoring of the Effects of the Neurological and/or Muscular Stimulus

In one embodiment, after the muscular and/or neurological stimulus is applied, the behavioral response of the fish is monitored. Behavioral responses measured as indicative of a seizure include 1) rapid and erratic movements and 2) post-ictal inactivity. This approach allows measurement and quantification of a fish's behavior during and following a seizure. This post ictal rest period is reproducible in duration and indicative that a seizure has occurred. Agents that block the production of a seizure produce a very active fish following stimulation that does not show typical seizure activity and does not exhibit the post-ictal somnolance. An optical approach entails the use of a light beam between an LED emitter and detector (including photodiode, phototransistor, photodarlington, photomultiplier) pairs, which is disrupted by the animals' movement in the well, or a CCD/CMOS based camera.

Good signals for detection of fish movements can be obtained with either reflectance emitter detector modules (usually situated on the bottom of the well) or transmittance modules passing through the wells. In order to monitor the fish's motions, a wavelength of light that is not visible to the fish may be employed. Teleosts are able to see light up to about 670 nm, have a 570 nm centered rhodopsin, and appear to be sensitive down into the ultraviolet. Ultraviolet or infrared light may be employed. In one embodiment, infrared (NIR) light is employed. Illumination can be provided by any of a variety of light sources, such as incandescent lamps, hollow cathode lamps, gas vapor lamps, light emitting diodes, lasers, semiconductor diode lasers, tunable dye lasers, and the like. Light emitting standard diode packages with emission wavelengths in the NIR range in wavelengths from 650-900 nm are available. At wavelengths above 800 nm, good signals could be obtained, but the fishes' tails may become almost transparent to this wavelength range of light, making subtle movement less detectable. Therefore, in one embodiment, the 700-800 nm range is employed. Transmittance as opposed to reflectance detection may be more sensitive.

Detection of neuromuscular response to painful stimuli evoked by mechanical or chemical means is afforded by either optical sensing devices as described above for seizure detection or by recording electrical signals evoked by musculature contraction or neuronal activity. Recording of electrical activity can be made either by electronic detection of local currents, magnetic field alterations, optical via voltage- or calcium-sensitive dyes or fluorescent protein based constructs. Fluorescent signals are measured by industry standard fluorescent stereo or compound microscopes or illumination with a specific wavelength light and inclusion of a emission filter on the front of the camera or light sensitive array.

Cardiovascular signals are recorded either by standard black and white or color video cameras used to directly image the heart through the transparent animal body of the younger animals. Direct observation is also possible in adult teleost that have been specifically breed to be transparent. Analysis of sequential frames of time video time series is employed to extract various cardiac parameters such as heart rate, QT interval, AV nodal block etc. For greater contrast transgenic animals can be used which express fluorescent proteins specifically in cardiac tissues.

Detection

The detection means may include, but is not limited to the following:
i) a plurality of photodetectors situated substantially adjacent to a side of a test well, and a plurality of photoemitters situated substantially adjacent to a side of a well substantially opposing the plurality of photodetectors;
ii) electrodes present within at least one test well capable of detecting electromagnetic impulses generated by the motion of the fish in the well;
iii) a microphone or a piezoelectric sensor present within at least one test well, wherein the microphone or piezoelectric sensor is capable of detecting electromagnetic impulses generated by the motion of the fish in the well;
iv) a video camera, a CCD-based imaging system, or CMOS-based imaging system capable of recording the motion of the fish in the well;
v) magnetic sensing coils located parallel to the axis of the test well;
vi) magnetic sensing coils which are located perpendicular to the axis of the test well;
vii) multiple magnetic sensing coils located both parallel and perpendicular to the axis of the test well; wherein the signal in the coils is quantified as the difference between the signals in the parallel and perpendicular coils;
viii) multiple magnetic sensing coils located both parallel and perpendicular to the axis of the test well; wherein the signal in the coils is quantified as the first derivative of the difference between the signals in the parallel and perpendicular coils; or
ix) a superconducting quantum interference device (SQUID) magnetometer located proximally to the wells.

In some embodiments, detection is performed in real time.

A number of detector options (i.e. photodiodes, photoresistors, photodarlingtons and phototransistors) exist. A small array (n=4) of photodiodes covering the bottom of a test well provides a good signal. A high speed camera is also effective detector that may be used in this invention.

When a photo diode array is employed, the detection means fore each well may comprise a plurality of photodetectors, wherein the number of photodetectors is at least four, and is optionally six, eight, ten, twelve, fourteen, sixteen, eighteen, or twenty. From 50 to 70 total discrete photodiodes are typically present in the photodiode array. However photodiodes can be made in arrays to physically match each well. In certain embodiments, the photodiode is a fiber optic bundle with a flat end that is flush with the well bottom. In this embodiment, the bundle breaks up into discrete fibers that each connect to a photodectector on a PC board. Using readily available 1.2-micron technology, photodiodes on a 20-micron grid can be made 10×10 array, providing 100 photodiodes in an area only 0.04 mm², i.e., about 2500 photodiodes per mm². Also a honeycomb array of 61 elements can be used as a dot image a single well in a 48-well plate. Higher photodiode density is achievable using commercially available 0.5-micron processes. In other embodiments the array may be a 1000 element array capable of fitting on a 5 mm×6 min IC, a medium-sized die format.

The photodector may comprise a photoresponsive electrode. In some embodiments, the photoresponsive electrode is a semiconductor electrode from which an electrical signal is inducible or variable, depending upon the effect of irradiation. The photoresponsive electrode(s) may be made of a semiconductive material, such as silicon, gallium arsenide, gallium selenide, aluminum gallium arsenide, and the like, and combinations comprising one or more of the foregoing materials. The semiconductive material may comprise either a p- or n-type semiconductive material, and, as appropriate, may be intrinsic or may employ such dopants as boron, aluminum, phosphorus, arsenic, antimony, and the like, and combinations comprising one or more of the foregoing materials. The degree of doping may be varied widely, there being a wide variety of commercially available doped wafers may be employed. The concentration of the dopant will normally vary empirically in order to provide the desired photoresponse, frequently being a matter of convenience, and may be about 10¹⁰ to 10²⁰ atoms/cc; usually for silicon the rating will be about 5-20 ohm-cm.

Other methods of physiological monitoring may be employed. These include, but are not limited to:
i) monitoring the fish tail movement, which is rapid, by means of a microphone or miniature piezoelectric sensor placed in each well;
ii) administering to the fish magnetic particles, which produce a large electromagnetic signal, and monitoring the magnetic field generated in the well by the fish;
iii) monitoring of the magnetoelectroencephalogram of the fish in the well; and
iv) monitoring of the electrocardiographic or electromuscular patterns of the motion of the heart or trunk musculature of the fish in the well.
   Signal Processing
   Optical Detector and Amplifier

Signal processing may include an optical detector and amplifier circuit. This detector may be implemented on an integrated circuit in order to convert an optical signal into an electrical signal suitable for data digitization and capture by a computer. In one embodiment, the optical detector and amplifier include a phototransistor coupled to a transimpedence amplifier, which converts a current signal into a voltage signal and which is followed by an amplifier. The operational amplifier in the transimpedence amplifier may be a two-stage, unbuffered amplifier.

In one embodiment, a capacitor has a value of 2 pF and resistor has a value of 100 KΩ. The gain of amplifier is equal to 1+(Resistance X/Resistance Y). Thus, in one specific embodiment for which the gain of the amplifier is 10, resistors X and Y are chosen so that their ratio is 9. The t is designed to be useful for wide-band amplification and low-level signals. The gain-bandwidth product is 70 MHz, and the amplifier is stable for gains greater than 10. In addition, the circuit has an input offset voltage less than 5 mV, a DC gain of 220, a positive slew rate of 80 V/μs, and a negative slew rate of 9 V/μs. The circuit may include 2.5 mW from a single 5 V supply.

The circuit may be modified as required by specific applications. Since the phototransistor cell is composed of basic photocell elements, it may be connected to as many cells as needed to create a desired geometry or a required number of channels needed to adapt the detector to a specific application. Other light sensing structures in addition to the phototransistor may be fabricated using standard CMOS processing steps. Several photodiode structures are possible using standard p-n junctions.

Although this embodiment has illustrated one method for measuring low current levels from a phototransistor, many other methods are possible. For example, one method of determining the current is to integrate the current using an integrating amplifier for a fixed time and then measure the voltage or to integrate the current until a fixed voltage is reached and then measure the time. A second method to determine the current would be to use an oscillator. In particular, an integrator could continually integrate the unknown current until a preset voltage was reached and then reset itself. The resulting frequency of oscillation would be proportional to the current. In addition to using standard capacitors, one could implement this second method using the capacitance of the photodiode itself. Initially, the switch is closed and the capacitance of the photodiode is discharged and there is zero voltage across the photodiode. When the switch is opened, light impinging on the photodiode creates charge, which produces a voltage on the photodiode capacitance. More light increases the voltage, which is amplified by the amplifier. When the output of the amplifier exceeds the reference voltage, the output of the comparator changes state, firing the one shot which in turn closes the switch and discharges the photodiode capacitance. This resets the amplifier input to the initial state and the comparator also returns to its initial state. When the one-shot times out, the switch opens and the process of charging can start again. A greater light level results in faster charging and therefore a higher frequency output of the oscillator. This results in an array of integrators that could be multiplexed to a single fast amplifier without the loss of significant measurement time.

Signal processing may utilize two 64 channel A-to-D conversion devices assembled from National Instruments hardware. This system will digitize two signals from each well; the activity signal and the application of either the chemical or electrical convulsant. Each of these signals may be digitized at around 1000-2000 samples per second. Using LabView programming, activity within wells can be assayed prior to stimulation to ensure adequate activity. Upon reaching a point of adequate spontaneous activity, the system may automatically deliver the muscular and/or neurological stimulus and the nature and duration of the seizures and post ictal inactivity, for example, can be measured. The digitized signal may be thresholded and fed into a TTL one shot multivibrator which feds a audio monitor (speaker). Monitoring the movements as sound allows comparison of visible signal we see on a video monitor with the sound of the animals movements.

Other more sophisticated signal detection algorithms may be employed to increase the signal to noise ratio. These include fast fourier transformation of the summed signals allowing conversion from the amplitude domain to the time domain; amplitude discrimination analysis using a software pulse height analysis system, which is a simple extension of the threshold detection system actually employed in obtaining the results supra; and a parallel processor based, trainable, neural network model for signal elucidation. All of these methods and implementation of the associated algorithms are well known to those skilled in the art of signal detection and extraction.

The final prototype device comprises a 96 position array of photodetectors, wherein each position contains a plurality of photodetectors, wherein the number of photodetectors is at least about four, and is optionally six, eight, ten, twelve, fourteen, sixteen, eighteen, or twenty, but typically not greater than 128, and is most optimally in the range of eight to twelve. This device contains the associated amplification and integration hardware in a single printed circuit board. This device integrally comprises and supports a multiwell plate to which a second device containing a similar array of photo emitters and electrical stimulators will be secured atop of the plate in a sandwich. A plurality of such devices may be optionally employed as part of the present instant invention.

Other types of multiwell plates may be employed, for example those containing 1096 wells, 384 wells, 48 wells, 24 wells, 12 wells, and so forth. The present examples are nonlimiting with regard to the plurality of wells in the particular design. Larger number of wells may require smaller optical detection components, but such components of reduced size are readily commercially available and can be constructed by one well versed in the art. For example, such components of reduced size are commonly used in the production of inexpensive digital cameras for the consumer market.

Exemplary Embodiment of a Device for Production of an Electrical Stimulus and Detection Of a Physiological Response

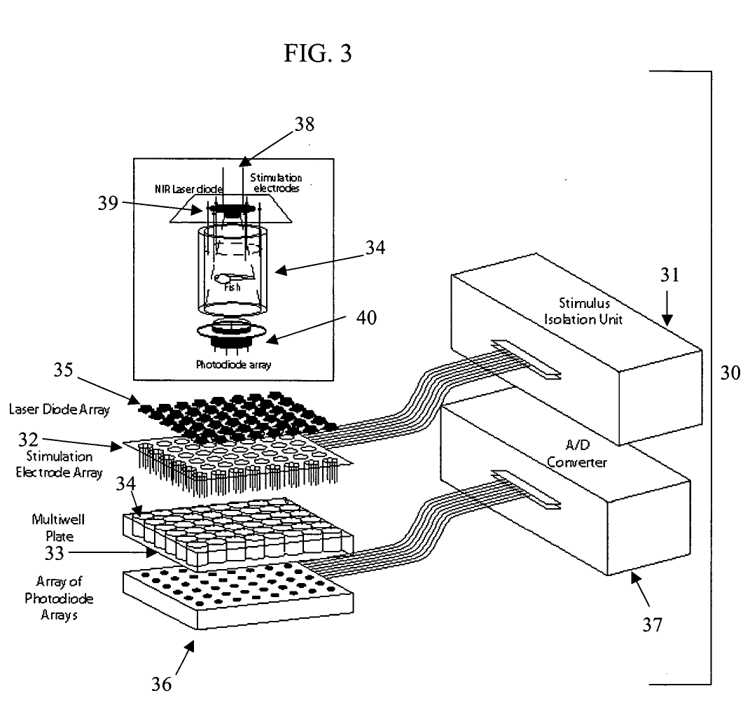

FIG. 3 shows an exemplary embodiment of a unit (30) for production of an electrical muscular and/or neurological stimulus and detection of muscular and/or neurological activity. In this specific embodiment, a Stimulus Isolation Unit (31) is in electrical communication with a Stimulation Electrode Array (32) which is in turn in communication with a 48-well multiwell plate (33). The Stimulation Electrode Array (32) provides an electrical field to induce a muscular and/or neurological activity in a teleost fish located in a well (34). The Stimulation Electrode Array (32) is arranged such that a bundle of electrodes is available to contact each well (34) of the multiwell plate (33).

A laser diode array (35) is also located above the multiwell plate (33) such that a near-infrared laser diode is available to illuminate each well (34) of the multiwell plate (33). In operation of this embodiment, the laser diode array (35) is in communication with the multiwell plate (33). In addition, an array of photodiodes (36) is located below the multiwell plate (33) in communication with (e.g., to substantially oppose) the array of near-infrared laser diodes (35) allowing detection of both the light transmitted through each well (34) and the application of the electrical field in each well. This photodiode array (36) transmits the detected signals to an attached A/D converter (37) such that the detected signals can be digitized.

The inset shows a close-up view of an example of a well (34) comprising a teleost fish according to an operation of this embodiment. A stimulation electrode bundle (38) of the Stimulation Electrode Array (32) is in contact with the fluid in the fish-containing well (34). Additionally, a near-infrared laser diode (39) is in communication with the top of the well (34) to illuminate the fish-containing well. On the opposite side of the well (34), a photodiode (40) is in communication with the bottom of the fish-containing well (34). In this manner, a stimulation electrode bundle (38) is in electrical communication with the fish-containing well (34), the well further being in communication with a laser diode (39) and a photodiode (34) such that the behavioral response induced by the stimulation electrode bundle (38) can be detected.

### EXAMPLES

### Example 1

### Fish Maintenance

All fish are about the same age and have been maintained in substantially identical physiological environments. Teleosts are highly responsive to light, therefore fish are maintained on a constant 12/12 hr light-dark cycle. The temperature of the aquarium is maintained at about 40° F. with a chiller. Well-water (non chlorinated) is employed to propagate and raise the fish. Temperature, pH, and ionic composition of the well water are monitored continuously.

### Example 2

### Acclimation

The plate is placed in a darkened room and secured on a diffuse light table. The light table uses infrared LED arrays and is placed on an antivibration table. The light table is in a room with reduced ambient light and sound. The plate is placed in the sensing device and secured in the darkened room and the LED array illuminated. The animal remains in the well for an acclimation period (10-30 minutes) in the darkened quiet room. Following the acclimation period, the animal's activity in the wells is recorded for a given period of time. This period represent the pre-drug control condition. Following the pre-drug period a solution containing the test agent and/or a chemical stimulus is added to the well.

### Example 3

### Induction of Convulsions

Two general methods of seizure induction are used: electrical activation via electrodes placed in electrical contact with water in the well and addition of chemical proconvulsant agents to the bathing water

1. Electrical Activation via Electrodes Placed in Electrical Contact with Water in the Well.

Consistent electrical-induced seizures are achieved by optimizing both maximum voltage achieved, overall power delivered during relatively constant current. Electrodes are placed in the wells from above just prior to commencement of the test.

For the electrically induced seizures an embodiment includes: Handling, distribution of fish and acclimation to test compounds as above. Convulsions are produced by the placing a circular array of 6 stainless steal wire electrodes (around 100 μm in outside diameter) around the perimeter of a single well in a 48 well plate. The electrodes are insulated to the tips on the side of the electrodes that face the inside of the well. In this way the fish is less likely to contact the metal of the electrode that faces the well wall. A seizure is evoked by the application of a constant duration pulse of a constant current sine wave oscillating voltage field. The amount of current applied depends on the electrical impedance of the arrangement. In general around 30 mA of current at around 70 RMS volts is sufficient to produce a reliable seizure in a 2-3 week old Medaka fish. During the pulse the output is randomly scrambled to opposing pairs of the six electrodes via a high voltage MUX circuit. The electronics to produce the above stimulation are apparent to person skilled at the art.

The application of sufficient electrical current to the water containing a fish will evoke a behavioral response. However, at a specific range of current/voltage settings (30-110 V; 20 mA-0.1 mA) an animal will have a seizure, experience a post-ictal period of quiescence and recover. At significantly higher voltages the animal will be killed and at lower doses, a full generalized tonic-clonic seizure will not be evoked. Application of a battery of classic anticonvulsants known to block seizure generation in animals will block the expression of seizures in teleost. Carbamazepine, felbamate, phenytoin (10-80 μM) and gabapentin all were capable of blocking seizure specific behavioral movements in teleosts following electrical stimulation.

Application of the electrical pulse is governed by a central computer.

2. Addition of Chemical Proconvulsant Agents to the Bathing Water

Proconvulsant agents are added to the well as a concentrated solution either a) by addition of escalating concentration in discrete steps (SupraThreshold Method) or continuous ramp increase (Ramp) in concentration to identify threshold for production of seizures.

### Example 4

### Introduction of Test Agents to the Wells

Test agents (1 mM-20 mM) in dimethyl sulfoxide (DMSO) are contained in barcoded multiwell plates (e.g., 96-, or 48-well plates). Aliquots of DMSO containing agents are either replated at diluted concentrations with tank water, or added directly to wells containing fish. Experimental agents are added manually or via an automated robotic liquid handling robot (i.e. Tecan). Test agents are added to different wells containing fish at escalating doses to achieve final concentrations between (1 μM and 100 mM) to obtain the effective dose (ED) of the unknown compound. Solubility of the added agent is estimated by comparing the turbidity (i.e. optical density) of the well solution before and after the addition of the compound. Addition of up to 10% DMSO to well water does not modify seizure responses or other muscular and/or neurological responses.

### Example 5

### Recorded Data for Induced Convulsions in Teleost Fish

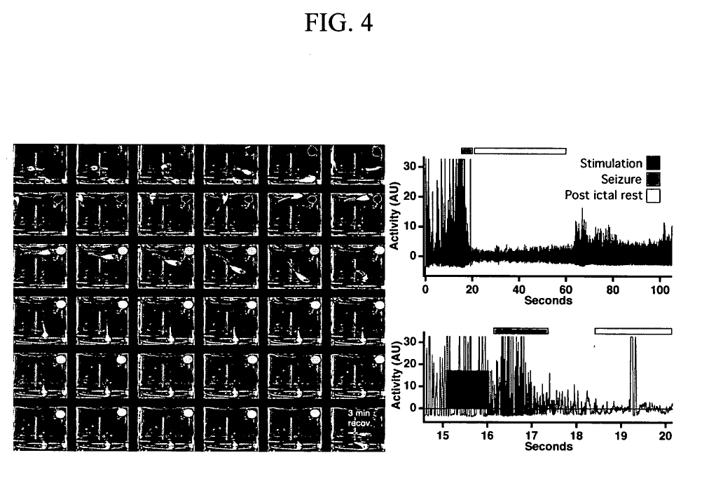

FIG. 4 shows an example of recording the induced convulsant activity in a teleost fish in real time. The left panel is a series of serial photomicrographs depicting the fish behavior before, during, and after induction of seizure activity. The fish motion is quantified as activity units (AU) and is graphed on a time line. In this example, a one-second electrical stimulation is applied to a teleost fish exhibiting normal behavior that induces a convulsant seizure that lasts for approximately 1.5 seconds. The convulsive seizure is associated with very rapid activity. Following the seizure, the fish enters a post-ictal rest phase which is associated with significantly reduced activity. This reduced activity phase may last for several minutes.

Fish behavior can be recorded over various time spans. The two graphs represent both long term and short term modification of the fish's behavior. The top graph shows the fish behavior during 100 seconds. In this graph, the post-ictal rest phase lasts for about 40 seconds following the induced seizure. The fish activity following this post-ictal rest phase is reduced compared to the pre-stimulation behavior. The bottom graph shows that the fish behavior may also be recorded over very short time intervals to provide a detailed account of the fish behavior during and immediately following an induced seizure.

### Example 6

### Screening Agents for Anti-Seizure Activity Using a Chemical Stimulus

Determining Convulsive Dose

To establish the minimum seizure threshold dose (CD₅₀) for convulsant agents 5 ml of tank water is placed a single well of a 12-well plate. A single juvenile teleost is placed in the well and allowed to acclimate for a period of time in a darkened room. When the animal has acclimated to the new conditions, a solution containing convulsant (e.g. a 20 mM solution of pentylenetetrazol (PTZ) in tank water) is added to the well at a rate of 0.1 ml per minute. The amount of solution being added is constant. The behavior of the fish is then monitored continuously during the rest period beginning with the addition of the convulsant. At the point which the animal has a convulsion the addition is stopped and the animal removed. This terminal concentration for PTZ was found to be around 6 mM for zebrafish and higher for Medaka (10 mM). However immersion of adult zebrafish into 5 mM of PTZ was found to evoke a seizure 100% (10 fish) of the time within 30 minutes of addition. This dose is considered the convulsive dose (>97%) for teleost.

Dosing Test Agents Prior to Evaluation

Test agents are added to the well at doses from 1 mM to 100 mM 30 minutes prior to the convulsant test. Several anticonvulsants are tested for activity in the PTZ test; valproic acid (500 μM-10 mM), felbamate, gabapentin, phenobarbital and ethosuximide. Administration of compounds in the water causes absorption in fish through both the gills, orally, and through the skin. Occlusion of the esophagus reduces the amount of compounds absorbed by some 30 percent indicating the extent of absorption via the gills and skin. Lipophilic compounds, like many anticonvulsants are absorbed better through the gills and become partitioned into the fish over time. Thus dosing of test compounds can be extended for days prior to testing.

One embodiment of the screen for anticonvulsant activity in the presence of a chemical stimulus includess placing a single or plurality of adult (1 month) fish into each well of a 12 well plate. This well contains 4 ml of pure tank water with a defined pH (7.0 can be between 6-8), hardness (90 ppm). The test compounds are then applied to the wells in 12 concentrations ranging from 10 μM to 10 mM. The compounds are diluted from standard DMSO stock solutions stored in other multiwell plates. Test compounds are added as either tank water diluted solutions or as DMSO concentrates. Up to 5% DMSO can be added without significantly affecting the fish over a short period (3 days). The compounds are added to wells after the optical clarity (transmittance) of the well water is determined. Following addition of the compounds to the wells a second transmittance measure is made to determine if the compound has dropped out of solution at any of the concentrations. Those wells that exhibit significant decreases in transmittance are excluded from the analysis. If a compound is not soluble at 10 μM a new plate of fish is used diluting below this concentration until solubility is achieved. Highly insoluble compounds are solublized in cyclodextrins or mixed with wet fish food, dried and applied at a known amount to wells containing a fish that was not fed for a day.

Plates containing soluble compounds are covered with plastic tops from the manufacturer and placed in an incubator set around 27 degrees centigrade and an oxygen rich atmosphere. After an incubation period with the test agent of up to 2 days, the fish are tested for anticonvulsant activity. Immediately prior to testing the water containing the test compound is removed and replaced with fresh water to avoid particulate (food, feces, undissolved compound) interference in the behavioral measurements. Fish are examined by the operator for deaths and they are scored as such.

Evaluation of Test Agents

Testing is performed in a visible light darkened, sound dampened room upon a highly diffused infrared light box. A high-speed video camera is focused on the plate at a defined distance to produce an image of precise size. The plate is allowed to remain in the dark for 30 minutes prior to commencement of the experiment. At which point images are collected of the fish's movements with only of the test agent present. Excess or erratic activity is automatically determined by a computer algorhythim during a 20 minute control period. Following this period a concentrated solution of PTZ (20 mM in tank water) is applied to all of the wells simultaneously to a final concentration of 6 mM (for adult Medaka; 5 mM for adult zebrafish). Images are collected (at between 30 and 200 frames per second) for the next 30 minutes. At which point the animals are removed from the wells and placed in a common tank for removal from the laboratory.

The image sequence collected following the administration is examined by a computer program which determines a number of factors about the activity of the fish including orientation, location, distance traveled, and cross sectional area of the fish's silhouette. This information and other processed versions of the images are examined for a principle component of change. Each well is examined separately by the program. Activity levels reaching specific thresholds and the type of activity (circular swimming and erratic darting) are scored along with periods of quiescence. Periods of high-speed erratic and circular activity lasting more than 3 seconds followed by a period of quiescence or ‘shaking’ are scored as a generalized seizure. The latency to first seizure and the magnitude and frequency of seizures are scored for each well. Such dilution plates are repeated at least 6 times to ensure accurate results. From these dilution curves a rough ED₅₀ is generated for each test agent that demonstrates anticonvulsant activity. Compounds (test agents) that show acceptable potency are examined in additional experiments in which more fish are tested at greater numbers of concentrations to produce a more accurate ED₅₀ estimation.

In addition to PTZ, penicillin, strychnine, picrotoxin, bicuculline, kainate, pilocarpine or any of a range of pro-convulsant drugs, or any combination thereof can be used a pro-convulsant chemical stimulus in the above assay. These pro-convulsant drugs include blockers of GABAergic and glycineric neurotransmission and activators of glutaminergic neurotransmission.

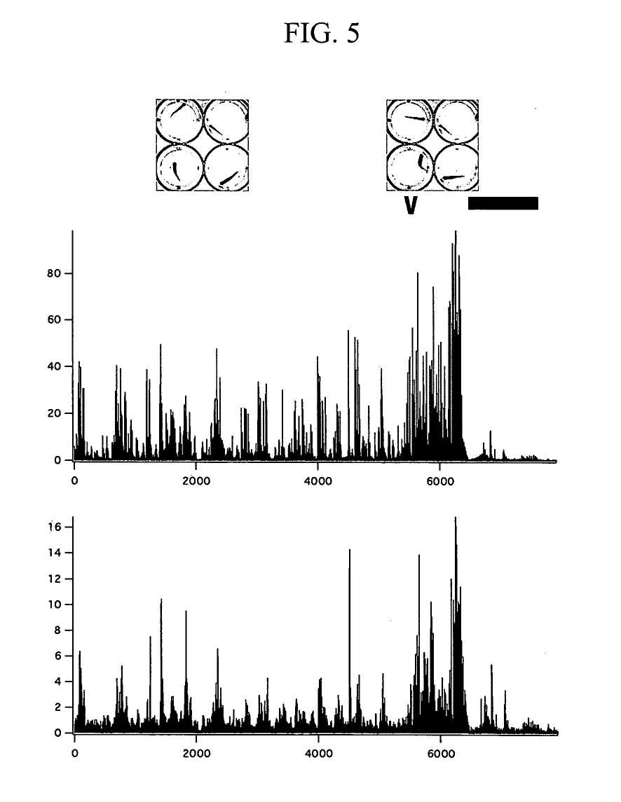

FIG. 5: Shows images of four wells with fish. Lower left well contains fish with convulsant (6 mM PTZ). Lower right contains fish with convulsant (6 mM PTZ) and anticonvulsant (Valproate, 5 mM). Upper left well is only tank water and upper right well is only valproate, 5 mM. Only the fish in the lower left well has seizure. The seizure is seen in the traces (middle distance traveled per frame and lower trace indicates change in orientation). Seizure detected by computer begins at V. Bar indicate post ictal rest period.

### Example 7

### Screening Agents for Activity as Pain Modulators

Following the administration of a test agent and an appropriate time to achieve dosing as described in Example 6, the animals movements are tracked by either an array of photosensitive elements above or below the well or a camera or other imaging device. At a specific time dictated by a computer program controlling the experiment, a laser is focused on the tail of the animal. The tail of the animal is determined by an image analysis computer algorithm which identifies the animal in silhouette in real time. A laser beam reflected from a mirror attached to an XY scanning mirror targets the animal's tail. The beam is targeted to a predefined location on the tail in relation to the head (i.e. ⅓ or ⅔ caudally from the head). Once the targeted location is acquired and the animal is deemed in a state of little motion (i.e. very little swimming motion) the laser beam intensity is linearly increased in power over a short (around 10 second) period of time. The highest intensity is set at a point which will produce brief damage to the animals tissue. A range of visible, infrared and UV lasers can be used. Commercially available lasers contained in photoablation microscopes set ups are sufficient. The intensity levels are validated on animals not treated with test agents (controls) verses animals treated with classic anti-nociception agents (morphine, codeine, MS-222, lidocaine etc).

### Example 8

### Comparison Methods for Determining Anti-Convulsant Activity

Validity of the method provided by this invention by assessing test agents identified by the assay as having anti-seizure efficacy in a conventional assay of seizure activity

Hind Limb Tonic Extension Model

Antiepileptic activity is measured by assessing a compound's ability to prevent the hind limb tonic extension component of the seizure in groups of mice induced by maximal electroshock (MES) after oral or intraperitoneal administration according to the procedures of the Epilepsy Branch, NINCDS, and compared to the standard agents dilantin and phenobarbital. Activities (ED₅₀'s) in the range of 10-400 mg/kg after oral administration were obtained.

The Maximal Electroshock Seizure Test (MES),

In this assay corneal electrodes primed with a drop of electrolyte solution (0.9% NaCl) are applied to the eyes of the animal and an electrical stimulus (50 mA for mice, 150 mA for rats; 60 Hz) is delivered for 0.2 second at the time of the peak effect of the test agent. The animals were restrained by hand and released at the moment of stimulation in order to permit observation of the seizure. Abolition of hind-leg tonic-extensor component (hind-leg tonic extension does not exceed a 90° angle to the plane of the body) indicated that the compound prevents MES-induced seizure spread.

Subcutaneous Pentylenetetrazol Threshold Test (scMet)

In this assay the convulsant dose (CD97) of pentylenetetrazol (85 mg/kg in rats) is injected at the time of peak effect of the test agent. The animals are isolated and observed for 30 minutes to determine whether seizures occur. Absence of clonic spasms persisting for at least five seconds indicates that the test agent elevates the pentylenetetrazol induced seizure threshold.

Rotorod Ataxia Test

Acute anti-convulsant drug-induced toxicity in lab animals is usually characterized by some type of neurological abnormality. In mice, these abnormalities can be detected by the rotorod ataxia test, which is somewhat less useful in rats. In the rotorod ataxia test, neurological deficit is indicated by the inability of the animal to maintain equilibrium for at least one minute on a knurled rod rotating at 6 rpm. Rats were examined by the positional sense test: one hind leg is gently lowered over the edge of a table, whereupon the normal animal will lift the leg back to a normal position. Inability to return the leg to normal position indicates a neurological deficit.

What is claimed is:

1. A method of identifying an agent that modifies a muscular activity, a neurological activity, or both comprising:
contacting a teleost fish with a muscular stimulus, a neurological stimulus, or both, and a test agent;
detecting the muscular activity, the neurological activity, or both in the teleost fish;
wherein when the test agent produces a detectable change in the muscular activity, the neurological activity, or both in the teleost fish, identifying the test agent as an agent that modifies the muscular activity, the neurological activity, or both.

2. The method of claim 1, additionally comprising
contacting a second teleost fish with a muscular stimulus, a neurological stimulus, or both;
detecting a second muscular activity, neurological activity, or both in the second teleost fish;
wherein when the test agent produces a detectable change that is a difference between in the muscular activity, the neurological activity, or both in the teleost fish contacted with the test agent and the second teleost fish, identifying the test agent as an agent that modifies the muscular activity, the neurological activity, or both.

3. The method of claim 1 or 2, wherein the muscular stimulus, the neurological stimulus, or both, comprises an electrical stimulus, a chemical stimulus, or both.

4. The method of claim 3, wherein detecting the muscular activity, the neurological activity, or both comprises detecting a behavioral response in the teleost fish.

5. The method of claim 4, wherein the behavioral response is rapid and erratic tail movements, or post-ictal inactivity.

6. The method of claim 1 or 2, wherein the teleost fish is a Oryzias latipes, Astronotus ocellatus, Danio rerio, Anguilla anguilla, Chelon labrosus, Salmo truttafario, Oncorhynchus mykiss, Oreochromis mossambicus, Eigenmannia virescens, Cyprinus carpio, Stephanolepis cirrhifer, Carassius auratus, Gasterosteus aculeatus, Clarias batrachus, Pimephales promelas, or Apteronotus leptorhnchus.

7. The method of claim 1 or 2, wherein detecting comprising recording the muscular activity, the neurological activity, or both in real time.

8. A method of screening comprising:
providing a plurality of test wells, each test well comprising at least one teleost fish,
contacting the teleost fish in at least a first fraction of the test wells with a test agent,
administering a muscular stimulus, a neurological stimulus, or both to the plurality of test wells,
detecting a muscular activity, a neurological activity, or both in the teleost fish;
wherein when the test agent produces a detectable change in the muscular activity, the neurological activity, or both in the teleost fish in at least one test well, identifying the test agent as an agent that modifies the muscular activity, the neurological activity, or both.

9. The method of claim 8 additionally comprising
as a subset of the plurality of test wells, a second fraction of test well in which the fish in the second fraction of test wells are not contacted with a test agent,
wherein when the test agent produces a detectable change that is a difference between in the muscular activity, the neurological activity, or both in the teleost fish in the first fraction of test wells and the muscular activity, the neurological activity, or both in the teleost fish in the second fraction of test wells,
identifying the test agent as an agent that modifies the muscular activity, the neurological activity, or both.

10. The method of claim 8 or 9, wherein the detecting is performed in real time and the means for detecting the muscular activity, the neurological activity, or both is any one of the following:
a plurality of photodetectors situated substantially adjacent to a side of a test well, and a plurality of photoemitters situated substantially adjacent to a side of a well substantially opposing the plurality of photodetectors;
electrodes present within at least one test well capable of detecting electromagnetic impulses generated by the motion of the fish in the well;
a microphone or a piezoelectric sensor present within at least one test well, wherein the microphone or piezoelectric sensor is capable of detecting electromagnetic impulses generated by the motion of the fish in the well;
a video camera, a CCD-based imaging system, or CMOS-based imaging system capable of recording the motion of the fish in the well;
magnetic sensing coils located parallel to the axis of the test well;
magnetic sensing coils which are located perpendicular to the axis of the test well;
multiple magnetic sensing coils located both parallel and perpendicular to the axis of the test well;
wherein the signal in the coils is quantified as the difference between the signals in the parallel and perpendicular coils;
multiple magnetic sensing coils located both parallel and perpendicular to the axis of the test well;
wherein the signal in the coils is quantified as the first derivative of the difference between the signals in the parallel and perpendicular coils; or
a superconducting quantum interference device (SQUID) magnetometer located proximally to the wells, or a combination of any of the foregoing.

11. The method of claim 8 or 9, wherein the means for detecting the muscular activity, the neurological activity, or both comprises a plurality of photodetectors in communication with a plurality of photoemitters.

12. The method of claim 8 or 9 wherein the muscular stimulus, the neurological stimulus, or both is an electric field, a chemical stimulus, or both.

13. The method of claim 8 or 9, wherein the means for detecting the muscular activity, the neurological activity or both comprises:
a plurality of photodetectors capable of detecting seizure activity in the fish, wherein the photodetectors are situated substantially adjacent to a side of each well; and
a plurality of photoemitters situated substantially adjacent to a side of each well substantially opposing the array of photodetectors array; and
a means for recording the seizure activity in real time.

14. The method of any one of claims 1, 2, 8, or 9 wherein the muscular stimulus, the neurological stimulus, or both, is a convulsant stimulus, the detected muscular activity, neurological activity, or both is a convulsant activity, and the test agent inhibits the convulsant activity.

15. The method of claims 1, 2, 8, or 9 wherein the muscular stimulus, the neurological stimulus, or both is a neuropathic pain stimulus, the detected muscular activity, neurological activity, or both is a pain aversive activity, and the test agent inhibits the pain aversive activity.

16. The method of claim claims 1, 2, 8, or 9 wherein the muscular stimulus, the neurological stimulus, or both is a cardiac rhythm stimulus, the detected muscular activity, neurological activity, or both is a cardiac rhythm, and the test agent modifies the cardiac rhythm.

17. A screening system for identifying agents that modify a muscular activity, a neurological activity, or both comprising:
a plurality of test wells each test well comprising at least one teleost fish and at least one test well comprising a test agent,
a means for applying a muscular stimulus, a neurological stimulus, or both, wherein the means is sufficient to induce the muscular activity, the neurological activity, or both to the fish in the test wells; and
a means for detecting the muscular activity, the neurological activity, or both in the teleost fish.

18. The screening system of claim 17, further comprising a means for comparing the muscular activity, the neurological activity, or both in the teleost fish in the presence of a test agent to a control muscular activity, neurological activity, or both in the teleost fish in the absence of the test agent.

19. The system of claim 17, wherein the means for applying the muscular stimulus, neurological stimulus, or both is a pair of inert conductive electrodes capable of applying an electric field to the test wells.

20. The system of claim 17, comprising
a plurality of tests wells in the form of a multiwell plate,
a means for applying a convulsive stimulus sufficient to induce convulsive activity in the fish in the test wells, a means for applying a neuropathic pain stimulus sufficient to induce pain adversive activity in the fish in the test wells, or a cardiac rhythm stimulus sufficient to modify cardiac rhythm in the fish in the test wells
wherein the stimulus is either an electric field or a convulsive chemical.

21. The system of claim 17 or claim 20, wherein the means for detecting muscular and/or neurological activity in the fish comprises:
a plurality of photodetectors capable of detecting a muscular activity, a neurological activity, or both in the fish, wherein the photodetectors are situated substantially adjacent to a side of each well; and
a plurality of photoemitters situated substantially adjacent to a side of each well substantially opposing the array of photodetectors array; and
a means for recording the muscular activity, the neurological activity, or both, in real time,
wherein the muscular activity or neurological activity is a seizure activity, a cardiac rhythm, or a pain adversive activity.

22. The screening system of claim 17, additionally comprising a means for comparing seizure activity in the fish in the presence of a test agent to seizure activity in the fish in the absence of the test agent.

23. The system of claim 20, wherein the convulsive chemical is pentylenetetrazol, bicuculline, DMCM, FG 7142, strychnine, bemegride, 4-aminopyridine, kainate, NMDA, or any combination of the foregoing.

24. The system of claim 17, wherein the muscular stimulus, the neurological stimulus, or both is an electric field applied between alternating pairs of electrodes in an assembly in a sequential manner, and the average electric field experienced by the fish is substantially constant and substantially independent of the position of the teleost fish in the well.

25. The system of claim 24, wherein the photoemitters emit light not detectable by the fish, wherein the photodetectors are sensitive to this emitted light.

26. The system of claim 21, wherein the photoemitters emit light in a region of the electromagnetic spectrum not detectable by the fish, and the photodetectors are sensitive to light having this wavelength.

27. The system of claim 21, wherein the plurality of photoemitters emit light in the infrared or near-infrared region of the electromagnetic spectrum that is not detectable by the fish, and the photodetectors are sensitive to light having this wavelength.

28. The screening system of claim 17 or 20, further comprising a means for automatically adding reagents and fish to the wells.

29. The screening system of claim 21, additionally comprising a means for comparing the seizure activity in the fish in the presence of a test agent to seizure activity in the fish in the absence of the test agent.

30. The screening system of claim 17, wherein the test agents are analgesics.

31. The screening system of claim 17, wherein the test agents are agents to treat neurological diseases.

32. The screening system of claim 17, wherein the test agents are agents that are active on alpha- or beta-adrenoceptor subtypes.

33. The screening system of claim 17, wherein the test agents are agents that are active on dopamine receptor subtypes.

34. The screening system of claim 17, wherein the test agents are agents which ameliorate abnormalities in the Q-T cardiac interval.
